(19) United States
(12) Patent Application Publication
(10) Pub. No.: US 2023/0397866 A1
(43) Pub. Date: Dec. 14, 2023

(54) TEST TO DIAGNOSE, MONITOR OR STRATIFY DISEASES DIRECTLY OR INDIRECTLY ASSOCIATED WITH THE PATHOLOGIES OF THE CHOLINERGIC SYSTEM

(71) Applicant: Solvemed Group, Poznan (PL)

(72) Inventors: Hugo CHRÓST, Leszno (PL); Arkadiusz KLEMENKO, Poznan (PL)

(73) Assignee: Solvemed Group, Poznan (PL)

(21) Appl. No.: 17/916,125

(22) PCT Filed: Mar. 30, 2021
(86) PCT No.: PCT/PL2021/000020

§ 371 Date: Sep. 30, 2022

(30) Foreign Application Priority Data
Mar. 31, 2020 (PL) P.433391

(51) Int. Cl. A61B 5/16; A61B 5/00; G16H 50/20; G16H 50/70; G06N 20/00

(52) U.S. Cl. CPC A61B 5/163; A61B 5/4064; G06N 20/00; G16H 50/20; G16H 50/70; A61B 5/4082

(57) ABSTRACT

A test to diagnose, monitor or stratify diseases directly or indirectly associated with cholin ergic system pathologies, including Parkinson's disease, which involves a comparison of supplied pupil light reflex data against known normative data of the same kind, characterised by the fact that the supplied data contain the pupil light reflex data, which include at least one sample comprising at least one supplied parameter measured by a known device used to measure pupil light reflexes, and data which include at least one characteristic of the examined patient. The probability of disease occurrence is determined using machine learning algorithms, which comprise at least one neuronal network algorithm (SSN) and/or at least one mathematical function which is not a part of the neuronal network (SSN).

### RELATED APPLICATIONS

This application is a National Phase of PCT Patent Application No. PCT/PL2021/000020 having International filing date of Mar. 30, 2021, which claims the benefit of priority of Poland Patent Application No. P.433391 filed on Mar. 31, 2020. The contents of the above applications are all incorporated by reference as if fully set forth herein in their entirety.

### FIELD AND BACKGROUND OF THE INVENTION

The subject of the invention is a test capable of diagnosing, monitoring or stratifying diseases directly or indirectly associated with the pathologies of the cholinergic system, where the monitoring of such disease is possible through the pupil light reflexes (PLR), especially in patients with Parkinson's disease (PD), Alzheimer's disease (AD), Huntington's disease, Major depressive disorder (MDD) and other neurodegenerative diseases, in which direct or indirect pathologies of the cholinergic system occur. The diagnostic test can also apply to diseases such as the autism spectrum disorder, alcoholism, schizophrenia, and other diseases which may cause the pathology of the cholinergic system.

PD is a neurodegenerative disease. Prevalence of the disease varies between 41 per 100,000 people in the fourth decade of life to over 1,900 per 100,000 people aged 80 and older. It is estimated that in the United States alone the costs associated with PD regarding its management, social insurance, and lost earnings total at 25 billons annually. The cost of the medication alone is estimated at 2,500 USD per person, where the cost of a single operation can reach even 100,000 USD.

PD is a hyperkinetic syndrome (involuntary and excessive movement) characterised by muscle tremor, muscle stiffness, akinesia (difficulties with initiating movement), and bradykinesia (slowing down of movement). The most common type of PD is to a large extent non-specific (meaning that its onset is not apparent and its causes are not known). Rare types of PD may be genetically heritable. There are examples also of acquired PD which result from mechanical damage to the brain (midbrain) or through exposure to volatile toxins (e.g. pyridine). The abovementioned motor disturbances are caused by death of cells located in the compacted black substance (substantia nigra pars compacta, SNpc) located in the midbrain.

In healthy people, the number of neurons in the black substance (midbrain) decreases at a rate of around 5% per 10 years. A loss of about 50% of those cells (correlated with a 70-80% decrease in the level of dopamine in the striate body (corpus striatum)) is regarded as the onset of symptoms of PD, and so a natural decay in the level of those neurons can be a cause of PD only in the late stages of the disease. PET studies showed that in PD the rate of loss of the neurons increases dramatically (up to 12% per year). Thus, the changes causing PD begin around 5 years before the first symptoms appear.

Currently, no objective test (such as a blood test or EEG) exists which could enable a definitive diagnosis of PD. Instead, the doctor records a precise medical history of the patient and conducts a thorough neurological examination, looking especially for two or more cardinal signs (which are characteristic for the disease). Often, the doctor considers the patient's response to medication as another proof that the diagnosis is correct. However, early initiation of treatment may limit the patient's ability to participate in clinical studies, which urgently require newly diagnosed patients. In fact, the current hight average cost of drug development (about 2.6 Billion USD per approved drug) and the high failure rate of clinical trials in neurology (about 94%) represent major challenges in terms of financial sustainability and R&D productivity of biotechnology and pharmaceutical companies. Novel methods of reducing drug development costs and increasing clinical trial efficiency are urgently required. Some of the potential approaches that could improve this situation include diagnosing patients more accurately and at earlier stages of the disease to improve patient enrolment into clinical trials, stratifying patient cohorts using novel biomarkers to identify patient subgroup responding favourably to tested medicines, and providing companion diagnostic capabilities to identify in the real-world patients responding to specific treatments. In 2011, the Food and Drug Administration (FDA) approved a specialist imaging technique known as DaTscan, which allows doctors to capture precisely the dopaminergic system of the brain. This is the first diagnostic imaging technique approved by the FDA that can be used to assess movement disorders, such as those characterising PD. DaTscan on its own cannot diagnose PD, however, it can confirm the clinical diagnosis made by the doctor—something, that was not possible before. Around 26% of PD diagnoses made by doctors turn out to be incorrect. From among the falsely diagnosed, 48% were treated for a disease that did not exist, 36% were given medications against that disease, 6% were operated, and another 6% were both treated with medicines and operated. Considering the lack of an objective diagnostic test for PD and the low effectiveness of detection in its early stages, it is necessary to develop methods to detect PD before the cardinal symptoms appear and with the effectiveness greater than 74%. There is a well-defined body of evidence indicating that cholinergic insufficiency is a significant factor of neurodegenerative diseases such as, inter alia, AD and PD, which are caused by acetylcholine (ACh) and dopamine insufficiencies, respectively. What is more, most of the studies where the ACh-dependent PLR parameters were analysed, showed significant differences between age-matched healthy patients and those affected by AD or PD. In several studies comparing pupillometry parameters between PD patients and healthy controls, differences in the latency (T1), amplitude (AMP), maximum velocity (Vmax) and maximum acceleration (ACmax) were observed. However, there was no difference between healthy controls and PD patients in terms of the initial pupil radius (R1) and the minimal pupil radius (R2). Interestingly, the interpretation of the PLR results represents a significant challenge. There are numerous factors which have varying effect on the pupillometric values measured during an examination of the PLR. These factors include: the patient's sex, age, ethnicity, clinical information, genetic characteristics, eye colour, the equipment used, the level of adaptation at the start of the measurement, the stimulus characteristics, calibration details of the equipment, type of camera used, the temporal resolution, the spatial resolution, among other. This shows how challenging it may be to normalise of all the parameters when working on the diagnostic method which is to make use of this reflex.

The document U.S. Pat. No. 10,034,605 B2, presents the systems, equipment and methods, which can be used to detect and determine the extent of a brain trauma, mental incapacity, physical incapacity or other brain dysfunction through the measurement of the PLR, whereas the reflex in a patient not affected by a trauma can be regarded as “normal”, however, when a patient suffers from a brain trauma, the patient's pupil cannot constrict or dilate at the expected speeds and the constriction or dilation modes in response to the amount of light reaching the pupil, or can constrict or dilate at abnormal modes or speeds.

Prior art document US2012059282A1 disclosed only internet-based cognitive diagnosis system using visual paired comparison tasks. Document disclosed methods for diagnosing declarative memory loss using mouse tracking to follow the visual gaze of a subject taking a visual paired comparison test. It also disclosed methods for diagnosing dementia such as mild cognitive impairment (MCI) and Alzheimer's disease. The reported invention is relying on a visual paired comparison (VPC) task, which is a recognition memory task that assesses the proportion of time an individual spends viewing a new picture compared to a picture they have previously seen, i.e. novelty preference. The document teaches that there is a significant difference between patients with Mild Cognitive Impairment (MCI) and Normal Controls (NC) in overall preference for the novel images. It depicts the use of an eye tracker to precisely monitor subjects' eye movements.

Another prior art document US2018271364A1 disclosed a system for identifying abnormal eye movements includes a near-eye display (NED), an eye-tracking camera, a frame supporting the NED and the eye-tracking camera, and a processor in data communication with the NED, the eye-tracking camera, and a computer readable medium. This disclosure generally relates to devices, systems, and methods for identifying eye movement and pupil abnormalities. Documents teaches about stimulating and/or detecting ocular behaviour correlated to eye movement and pupil abnormalities. It also teaches that Parkinson's disease (PD) is the second most prevalent neurodegenerative condition.

After a brain trauma, it is possible to detect the occurrence and/or risk of brain damage, comparing the patient's pupils' reactions with the normal reaction of an undamaged pupil to determine whether the patient's pupil reaction was normal.

### SUMMARY OF THE INVENTION

The invention solves the problem of diagnosing diseases directly or indirectly associated with the cholinergic system pathologies.

The goal of this invention is to deliver effective, improved, reliable, non-invasive and safe diagnostic tests capable of detecting cholinergic system pathologies. Further goals of the invention are to deliver diagnostic tests capable of detecting pathologies of the cholinergic system at specific levels of sensitivity and specificity used for rapid testing as well as for professional laboratory tests. Further goals of the invention include delivering elements and sets used to move the test to diagnose cholinergic system pathologies, for example in the form of a mobile application. Than the goals of the invention include delivering machine learning algorithms capable of differentiating between disease subtypes, which, where such information is available, can be associated with particular drug treatments.

Those goals were achieved by developing a test diagnosing diseases directly or indirectly associated with the cholinergic system pathologies, based on a mathematical analysis, dependent on the cholinergic system and the pupil reflexes, especially to light.

The test to diagnose, monitor or stratify diseases directly or indirectly related to the cholinergic system pathologies, especially neurodegenerative diseases, including PD, which include a comparison of the PLR data is characterised, according to the invention, by the fact that it is completed by at least one computer device, whereas supplied data include the PLR data which include at least one sample containing at least one delivered parameter measured by a known device used to measure the PLR, and data about at least one characteristic of the examined patient, whereas if at least one empty value exists in the patient data, it is supplied by calculating a weighted mean and/or median and/or normal distribution of the data set, and then generated or captured data are identified and assigned to specific values, and resulting data are analysed by using at least one mathematical function by comparing measured values against standard data obtained from patients with the entry data adjusted to the direct or indirect pathology and/or healthy individuals in order to determine potential pathology in the analysed sample and the probability of occurrence of disease is determined using machine learning algorithms, which include at least one neural network algorithm (SSN) and/or at least one mathematical function which is not part of the neuronal network (SSN), and the resultant set of functions, comprising at least one mathematical function, includes a borderline point describing the probability of a disease occurring, whereas the borderline point is determined using the area under the curve ROC (receiver operating characteristic curve) and the effectiveness is calculated using the diagnostic test cross table (confusion matrix) and/or by evaluating the test sample (e.g. precision, F1-score, etc.). Beneficially, the determination of the diagnostic test result is completed through estimation of at least one assessment of a given cholinergic system pathology. Additionally, where the PLR data and data about at least one characteristic of the examined patient is taken from more than one patient, the machine learning algorithms are capable of using the supplied data from different patients to identify unique disease subtypes, which subsequently are used to assess the disease subtype of any newly examined patients, whereas the disease subtype is then associated with a particular drug treatment, where such information is available.

In the patent application, the term “sample” refers to a data set required to start a mathematical analysis conducted by an algorithm and to determine the diagnosis, by the term “patient characteristic” is understood any information describing factual physical or mental state that can exert an influence on the pupil reflexes. (For example, the colour of the iris, patient's age, sex, co-morbidities such as depression and similar diseases). While the term “parameter measured by the device to capture the PLR” includes all specially named data that can be captured using a hardware or software tool, such as: the latency of the onset of constriction, baseline pupil radius, minimal pupil radius, after the pupil reaction to light, amplitude, time to maximum miosis, which is defined as the time at which the constriction velocity is zero), maximum constriction velocity, maximum constriction acceleration, percentage amplitude, time to maximum constriction.

What is more, the included in this application technical data, documents, drawings, maps, projects, photographs, software, formulas, market analysis, as well as technical, technological and operational information, include parameters analysed by the invention, which are denoted only with an example symbol (e.g. T1) which does not bear any significance in the data analysis and can be replaced with any other symbol.

### BRIEF DESCRIPTION OF THE SEVERAL VIEWS OF THE DRAWINGS

The invention was presented in a drawing in which:

FIG. 1 shows the tree describing the functioning of the diagnostic mechanism;

FIG. 2. shows the comparison of part of the parameters considered at the examination and their comparison between healthy and ill individuals, and the analysis of the deviation from the healthy norm for those parameters;

FIG. 3. shows the ROC curve and the analysis of model functions describing the assignment to a disease with different borderline points;

FIG. 4. shows a scheme of main elements of basal nuclei (basal ganglia) and their elements.

### DESCRIPTION OF SPECIFIC EMBODIMENTS OF THE INVENTION

According to the model representation of the invention development, in order to create the diagnostic test, the averaged PLR values distributed according to the specifications of the Gaussian distribution were used, with 122 coming from PD patients and 101 from healthy controls.

### Example: Statistical Analysis of Data

The PD diagnostic test includes stages (a) supplying at least one sample containing at least one parameter measured by the device used to measure the PLR and at least one characteristic of the examined patient. (b) Used data are subsequently, or in parallel, subjected to cleaning where any empty values are substituted with average values from the training data set. In the case of categorical data, it is substituted with the most commonly occurring sample or by at least one other sample that could be significant in the diagnostic process. (c) Generated or acquired data are identified and assigned to specific values. With the help of a suitable tool, such as for example a pupillometer, through stimulation with light and/or other external stimuli, the R1 (initial pupil diameter) and R2 (minimal pupil diameter) parameters as well as the amplitude (R2-R1), maximum velocity (Vmax mm/s), maximum acceleration (ACmax mm/s\hat{ }2) and other parameters that can be calculated based on the video of the pupil constricting during its reaction to light, are obtained. Additionally, directly from the patient or from their medical record, information about their sex, age, race, prescribed medication and/or diseases which may influence the functioning of the diagnostic test, and any other information directly and/or indirectly related to the patient that may be significant clinically and/or technically is obtained. (d) Obtained data are analysed using at least one mathematical function.

For the purpose of calculation, deep learning multi-layer neuronal net is used and the error value is calculated based on at least one mathematical model, such as XGBoost, Logistic Regression or a deep learning neuronal net algorithm which consists of at least one hidden layer. All used models have the weight accuracy assigned based on the F1 score. The F1 score is calculated by multiplying by 2 the sensitivity/recall (the ratio between the true positive results to the sum of the true positive and falsely negative results), divided by precision/positive predictive value (equal to the proportion of true positive results among all positive results) divided by the sum of precisions and sensitivities.

F1_{score}=2×sensitivity×precision/sensitivity+precision

Such value will determine the error of each analysed model. (e) The resultant set of functions, comprising at least one mathematical function which includes a borderline point which describes the probability of disease occurrence. The borderline point is determined using the area under the curve ROC (AUC), and effectiveness is calculated using the cross table of the diagnostic test (confusion matrix) and/or by evaluating the tested data set (e.g. precision, F1 score, etc.) Currently, doctors use an assessment framework to determine presence of PD which includes:
1) Assessment framework based on identification of patient characteristics typical for this disease.
Sensitivity: 74%
Specificity: 48%
2) Following this invention, the framework of assessing the patient based on the algorithm using the PLR data. (Using a healthy control cohort of 45 individuals and 178 PD patients).
Sensitivity: 96%
Specificity: 94%

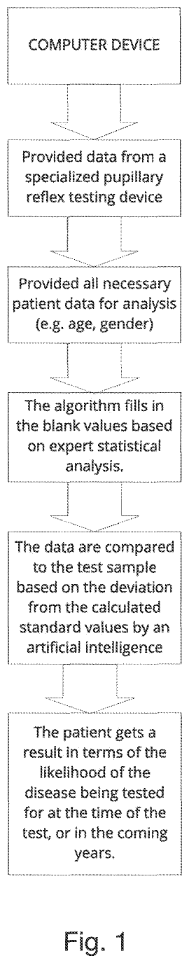

FIG. 1. shows the tree describing the functioning of the diagnostic mechanism, especially the sequence of actions completed by the algorithm during the diagnostic decision-making.

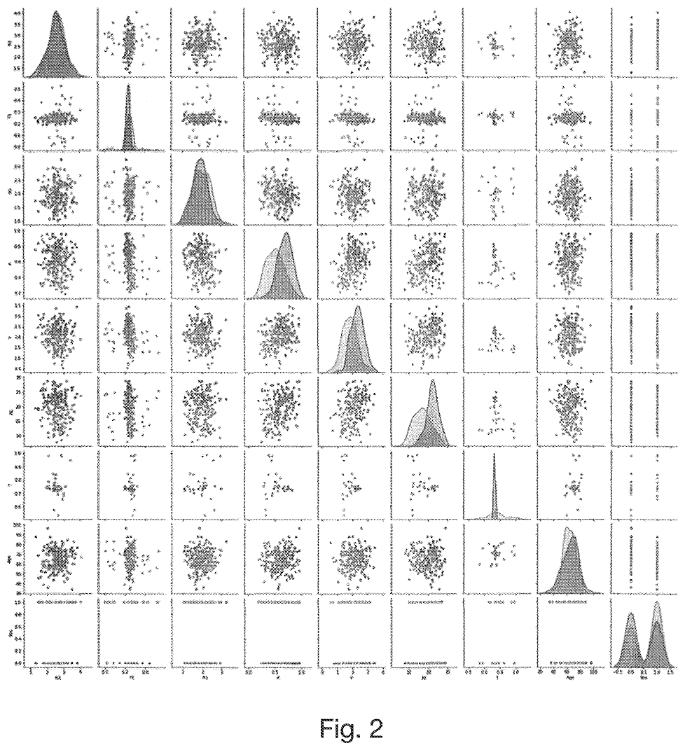

FIG. 2. shows the comparison of some of the parameters considered at the examination and their comparison between healthy and ill individuals, and the analysis of the deviation from the healthy norm for those parameters. In the figure, there is a comparison between 9 parameters. Light grey colour denotes ill individuals and black colour denotes healthy individuals.
Sex
Age
R1 (initial pupil diameter)
T1 latency
R2 (minimal pupil diameter)
Amplitude (R2-R1)
Maximum velocity (mm/sec)
Maximum acceleration (mm/sec\hat{ }2)
Time to maximal constriction

In the opposite corner, there is a distribution of samples from healthy and ill individuals for each parameter, while in individual squares there are samples from parameter from the x axis and the y axis, subdivided between healthy and ill individuals. The larger the distance, the stronger the relationship and more significant is given parameter at detecting the pathology of the cholinergic system.

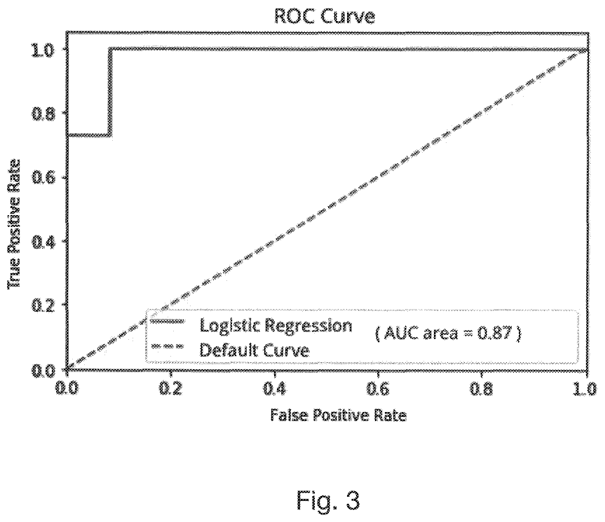

FIG. 3. shows the ROC curve and the analysis of model functions describing the assignment to a disease with different borderline points. The curve describes true values of the true positives for different borderline points of probability from 0 where all samples are assigned as negative to 1 where all samples are assigned as positive.

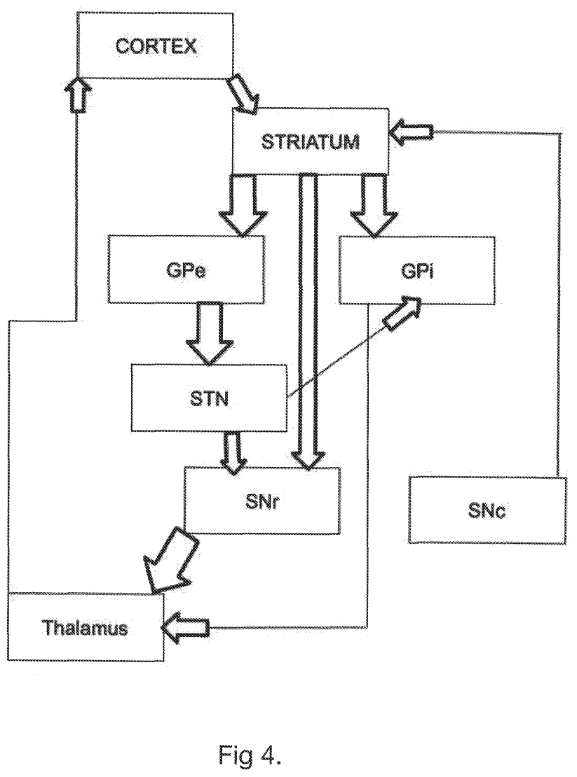

FIG. 4. shows a scheme of the main elements of the basal nuclei and their relative connections where the glutaminergic pathways stem from the thalamus to cortex, from cortex to stratum, from subthalamic nucleus to internal globus pallidus, from subthalamic nucleus to substantia nigra pars compacta to striatum, and where all the remaining pathways are GABAergic. Importantly, a significant loss of cholinergic neurons of the forebrain was confirmed in the brain affected by PD (Whitehouse et al., 1983; Candy et al., 1983). The identification of a larger loss of forebrain neurons in PD patients than in Alzheimer's patients made Arendt et al. (1983) suggest that deficits in the central cholinergic system may be as pronounced in PD as in AD. This explains the correlation between the neurodegeneration occurring in substantia nigra pars compacta in PD and the pathologies of the cholinergic system, based on which the diagnosis is made.

Table 1

Drugs which may affect the pupil relexes and their mechanism of affecting the reflex, Kelbsch C, Strasser T, Chen Y, Feigl B, Gamlin P D, Kardon R, Peters T, Roecklein K A, Steinhauer S R, Szabadi E, Zele A J, Wilhelm H and Wilhelm B J (2019) Standards in Pupillography. Front. Neurol. 10:129. DOI: 1.0.3389/fneur.2019.00129.

| | | |
|---|---|---|
| Drug | Mechanism | Pupil |

TOPICAL

| | | |
|---|---|---|
| Pilocarpine | cholinergic | miosis^{a} |
| Carbachol | cholinergic | miosis^{a} |
| Aceclidine | cholinergic | miosis^{a} |
| Atropine | anticholinergic | mydriasis^{b} |
| Scopolamine | anticholinergic | mydriasis^{b} |
| Tropicamide | anticholinergic | mydriasis |
| Phenylephrine | α₁-adrenoceptor agonist | mydriasis |
| Metoxamine | α₁-adrenoceptor agonist | mydriasis |
| Apraclonidine | α₁-adrenoceptor agonist | mydriasis^{c} |
| Dapiprazole | α₁-adrenoceptor antagonist | miosis |
| Brimonidine | α₂-adrenoceptor agonist | miosis^{d} |
| Cocaine | noradrenaline uptake inhibitor | mydriasis |

SYSTEMIC

| | | |
|---|---|---|
| Antihistamines | H1 histamine receptor antagonists | miosis^{e} |

ANTIHYPERTENSIVES

| | | |
|---|---|---|
| Prazosin | α₁-adrenoceptor agonist | miosis^{f} |
| Clonidine | α₂-adrenoceptor agonist | miosis^{g} |

ANTIARRYTHMICS

| | | |
|---|---|---|
| Disopyramide | anitcholinergic | mydriasis |

DRUGS FOR PARKINSON'S DISEASE

| | | |
|---|---|---|
| Anticholinergics | blockade of muscarinic receptors | mydriasis^{h} |
| Dopaminergics | stimulation of D2 dopamine receptors | mydriasisⁱ |

ANTIDEPRESSANTS

| | | |
|---|---|---|
| Tricyclic | mainly noradrenaline uptake blockade | mydriasis^{j} |
| Reboxetine | noradrenaline uptake blockade | mydriasis |
| Venlafaxine | noradrenaline/serotonin uptake blockade | mydriasis |
| SSRIs | serotonin uptake blockade | no effect^{k} |

ANTIPSYCHOTICS

| | | |
|---|---|---|
| Phenothiazines | α₁-adrenoceptor antagonist, sedation | miosis^{l} |
| Haloperidol | α₁-adrenoceptor antagonist | miosis |

SEDATIVES

| | | |
|---|---|---|
| benzodiazepines | GABA receptor agonist → sedation | no effect^{m} |

PSYCHOSTIMULANTS

| | | |
|---|---|---|
| Amphetamine | noradrenaline releaser | mydriasis |
| Modafinil | dopamine uptake blocker | mydriasisⁿ |

ANALGESICS

| | | |
|---|---|---|
| Opiates | stimulation of inhibitory μ receptors | miosis^{o} |

ANTIEMETICS

| | | |
|---|---|---|
| Scopolamine | anticholinergic | mydriasis |

ANTI-INCONTINENCE DRUG

| | |
|---|---|
| anticholinergic | mydriasis^{p} |

^{a}glaucoma treatment.
^{b}myopia treatment.
^{c}in Horner's syndrome (supersensitive α₁-adrenoceptors).
^{d}drugreduces noradrenaline release (glaucoma treatment).
^{e}first generation antihistamines (e.g. diphenhydramine, cyclizine) penetrate into the brain where they block H1 histamine receptors, leading to sedation.
^{f}drug blocks α₁-adrenoceptors in vascular smooth muscle.
^{g}drug stimulates inhibitor α₂-adrenoceptors on central noradrenergic neurones, leading to sedation and sympatholysis.
^{h}include orphenadrine, procyclidine, trihexyphenidyl.
ⁱD2 dopamine receptor agonists (e.g. pramipexole) stimulate inhibitory D2 receptors on wake-promoting centrl dopaminergic neurones, leading to sedation. This is expected to cause miosis, however, paradoxically, pramipexole causes mydrasis.
^{j}Tricyclic antidepressants block the uptake of noradrenaline, potentiating noradrenergic neurotransmission, and this would lead to mydriasis. However, they have some other effects: blockade of muscarinic cholinoceptors would lead to mydriasis and sedation, and blockade of α₁-adrenoceptors would cause miosis. The overall effect reflects the balance between these actions: mydrasis due to noradrenaline uptake blockade and cholinoceptor blockade in counteracted by miosis due to α₁-adrenoceptor blockade and sedation. This explains the variable effects of tricyclic antidepressants on the pupil: imipramine desipramine dilate it, while amitriptyline has little effect on it.
^{k}Selective serotonin reuptake inhibitor (SSRIs) block serotonin receptors in a complex network of serotonergic neurones associated with different excitatory/inhibitory receptors. The overall effect is little or no change in pupil diameter.
^{l}These drugs (e.g. chlorpromazine, trifluoperazine) also have anticholinergic effects that would lead to mydrasis. However, α₁-adrenoceptors blockade and sedation predominate, leading to miosis.
^{m}Paradoxically, alhough the benzodiazepine diazepam is highly sedative, it has no effect on pupil diameter.
ⁿModafinil blocks dopamine uptake at excitatory synapses on central noradrenergic neurones: this leads to increase in arousal and sympathetic activity.
^{o}Stimulation of inhibitory receptors on central noradrenergic neurones leads to sedation and sympatholysis.
^{p}These drugs (oxybutynin, festerodine) inhibit voiding of the urinary bladder by blocking cholinceptors in the detrusor muscle.

1-2. (canceled)

3. A method to diagnose Parkinson's disease, comprising comparing data for a patient comprising supplied pupil light reflex data comprising at least one sample of pupil light reflex data for said patient against a training data set comprising pupil light reflex data for known healthy and ill individuals, the method performed with the use of at least one computer device, wherein the patient data comprise pupil light reflex data comprising at least one supplied parameter measured by a known device used to measure pupil light reflexes, and the at least one characteristic of the examined patient comprising the patient's age and sex, wherein the method comprises, when at least one empty value occurs in the patient data, substituting the missing value with an average value from the training data set, and the pupil light reflex data is used to identify specific parameters comprising: the initial pupil diameter (R1), latency of the onset of constriction (T1), minimal pupil diameter (R2), amplitude (R2-R1), maximum constriction velocity, maximum constriction acceleration, and time to maximal constriction, wherein the method comprises analysing the patient data using one or more machine learning algorithms comparing measured values against training data acquired from patients with Parkinson's disease and healthy individuals in order to determine the probability of Parkinson's disease in the patient, wherein the machine learning algorithms comprise a logistic regression model, and wherein the method comprises determining a borderline point for the logistic regression model describing the probability of disease occurrence using a ROC, and wherein the method further comprises calculating the effectiveness of prediction of the probability of disease occurrence by the machine learning algorithm using a confusion matrix and/or by evaluating the precision and the FI score of the machine learning algorithm using a test sample.

4. The method of claim 3, wherein the specific parameters are selected from: initial pupil diameter (R1), latency of the onset of constriction (T1), minimal pupil diameter (R2), amplitude (R2-R1), maximum constriction velocity, maximum constriction acceleration, and time to maximal constriction.

5. The method of claim 4, wherein the specific parameters include all of: initial pupil diameter (R1), latency of the onset of constriction (T1), minimal pupil diameter (R2), amplitude (R2-R1), maximum constriction velocity, maximum constriction acceleration, and time to maximal constriction.

6. The method of claim 3, wherein the at least one characteristic of the examined patient comprises the patient's age and/or sex.

7. The method of claim 3, wherein the machine learning model comprises a logistic regression model.

8. The method of claim 3, wherein the specific parameters are determined using a pupillometer, through stimulation with light and/or other external stimuli.

9. The method of claim 3, wherein the specific parameters are determined using a video of the patient's pupil constricting during its reaction to light.

10. The method claim 3, wherein the method comprises: calculating the effectiveness of prediction of the probability of disease occurrence by a machine learning algorithm using a confusion matrix and/or by evaluating the precision and the FI score of the machine learning algorithm using a test sample.

11. The method of claim 3, wherein at least one parameter is derived from the pupil center position.

12. The method of claim 3, wherein at least one processing unit improves the pupil diameter measurement accuracy through at least one machine learning model.

13. The method of claim 3, wherein at least one processing unit improves the pupil diameter measurement accuracy through at least one mathematical operation such as deconvolution.

14. The method of claim 3, wherein a video of a patient is acquired from at least 40 cm distance enabling capturing of the patients face and at least one eye.

15. The method of claim 3, wherein at least one parameter is inferred from the conscious or unconscious eyeball movements.
